(19) United States
(12) Patent Application Publication
(10) Pub. No.: US 2018/0338867 A1
(43) Pub. Date: Nov. 29, 2018

(54) RESORBABLE PREPARATION FOR MEDICAL APPLICATIONS, AND CHARACTERISTIC USES FOR SAID PREPARATION

(71) Applicants: Tommaso Bianchi, Pianoro (IT); Federico Ponti, Bologna (IT)

(72) Inventors: Tommaso Bianchi, Pianoro (IT); Federico Ponti, Bologna (IT)

(21) Appl. No.: 16/051,821

(22) Filed: Aug. 1, 2018

Related U.S. Application Data
(63) Continuation of application No. PCT/IB2017/050537, filed on Feb. 1, 2017.

(30) Foreign Application Priority Data
Feb. 1, 2016 (IT) 102016000009733

(51) Int. Cl. A61F 13/02; A61F 13/00

(52) U.S. Cl. CPC A61F 13/0213; A61F 13/00012; A61L 27/24; A61F 2013/00693; A61F 13/0206

(57) ABSTRACT

The resorbable preparation for medical use, and in particular for use as an resorbable dressing for difficult wounds, includes an amorphous hydrogel composed primarily of a mixture of animal collagen, glycerol, xylitol and ozonized vegetable oil in water.

The mixture may include, depending on the applications and methods of preparation:
water, in proportion in the range of 10% to 90% by weight;
either animal or synthetic collagen, in proportion in the range of 40% to 70% by weight;
glycerol, in proportion in the range of 20% to 50% by weight;
xylitol, in proportion in the range of 0.05% to 5% by weight;
ozonized vegetable oil, in a proportion ranging between 0.001% and 10% by weight;
lactoferrin, in proportion in the range of 0.001% to 10% by weight;

The proportion of water is defined as a function of the desired consistency for the resorbable preparation.

### TECHNICAL FIELD

The present invention relates to the field of devices and products with high biocompatibility for medical use. In particular, the invention relates to a resorbable preparation suitable for different uses in the medical field, primarily for the treatment and the protection of deep or very extended wounds and the realization of devices for subcutaneous implantation.

Treatment of wounds is usually rather complex; this is in particular true for complex wounds, i.e. those that are not limited to lining tissues (skin and subcutaneous tissue) but that also affect the underlying structures (wings, muscles, tendons, nerves, vessels).

The occurrence of a wound in the body triggers a complex mechanism for tissue repair, that can be divided into two steps: an initial step, in which cells damaged are degraded by numerous leukocytes, which are concentrated in the site of injury (first 48-72 hours), while damaged proteins of the extracellular matrix are degraded by enzymes, such as metalloproteases; in a second step, also called proliferative phase, which is characterized by the accumulation of collagen fibers, produced by fibroblasts, and elastin. In absence of infectious events, the process leads to wound closure and formation of the scar.

Basically, the repair mechanism provides a strong proliferative activity promoting angiogenesis and new granulation tissue formation (a connective tissue, highly vascularized, composed of newly formed capillaries, proliferating fibroblasts and inflammatory cells); this activity gradually fills the wound. This step is followed by a remodeling step, which involves the maturation and organization of the fibrous tissue and that has broader implementation times, and it may take even several months to complete.

### BACKGROUND ART

Conventional treating techniques usually comprise deep cleansing of the wound and the subsequent application of an antiseptic dressing on the injured part. This dressing includes covering the injured area with permeable material, to ensure healing in a dry, or impermeable to water vapor, environment, in the case where a moist healing approach is applied. This latter approach is usually preferable, in that, it was shown that the creation and maintenance of an environment with the right humidity at the interface between the lesion and dressing significantly accelerates the healing process. Conversely, in the absence of such condition, the formation of crusts and drying of the tissues render the cell migration for the repair of the injury difficult and particularly expensive under the metabolic point of view.

It is also considered of fundamental importance, for the purposes of rapid healing, to prevent or delay as much as possible the replacement of the dressing during the wound repair process. In fact, it was shown that changing frequently the dressings lead to a cooling of the wound surface. This causes a consequent slowdown in recovery. In fact, cell proliferation reaches the maximum replication rate at a temperature of 35° to 37° C. Furthermore, premature or too frequent removal of adhesive dressings can lead to damage both in the surrounding skin (stripping of the epithelial cells) and in the bed of the injury.

In the case of deep wounds with significant removal of muscle tissue, it is also important that the dressing fills the wound cavity; the aim is to ensure the contact of the dressing with each area section of the injury. For the same reason, it is essential that the dressing be able to be shaped, so as to mate with the injured area surface.

Another treatment known as negative pressure wound therapy (NPWT) provides that the wound is filled with a spongy material and covered with a biocompatible plastic film, which adheres perfectly to the skin around the injured area. The interior of the wound is then connected, by means of a tube, to a vacuum machine, that creates a negative pressure within the covered area and sucks at the same time the excess exudate. It was shown that this technique allows great acceleration in healing of the wound.

The use of this treatment technique makes it even more important having a dressing that provides all the necessary aids to a proper and fast healing of a serious wound, and that does not need to be replaced.

Other fields and medical applications require availability of therapeutic devices conformable according to the needs, which are biologically active or that, otherwise, do not require to be removed once exhausted their therapeutic function.

Merely by way of example, as will become clear in the detailed description of the invention, this type of therapeutic devices is required for the application of temporary drains, or in the realization of the bowel or urinary ostomy management devices.

### OBJECTS OF THE INVENTION

An object of the present invention is to propose a preparation for medical use that is able to be so shaped as to meet the particular application requirements, also at the time of or immediately before use, in order to provide a functional, resorbable and biologically active support.

Another object of the invention is to propose a preparation for medical use that can assume consistency and durability, which can be defined based on the desired applications.

A further object of the invention is to propose the above preparation configured for use as a dressing for difficult wounds, also usable with negative pressure devices.

Yet a further object of the invention is to propose the above preparation configured for use as a resorbable catheter.

Yet a further object of the invention is to propose the above preparation configured for use as a resorbable plate for ostomy.

### SUMMARY OF THE INVENTION

These and other objects, which will become apparent from a reading of the detailed description of the invention, are achieved by a resorbable preparation for medical use, in particular for use as resorbable dressing for difficult wounds, which preparation includes an amorphous hydrogel consisting of a mixture of animal collagen, glycerol and vegetable oil ozonized in water.

The mixture may include, depending on the applications and methods of preparation:
water, in proportion in the range of 10% to 90% by weight;
collagen, preferably animal collagen, in proportion in the range of 40% to 70% by weight;
glycerol, in proportion in the range of 20% to 50% by weight;
xylitol, in proportion in the range of 0.005% to 5% by weight;
ozonized vegetable oil, in a proportion ranging between 0.001% and 10% by weight;
lactoferrin, in proportion in the range of 0.001% to 10% by weight;

The proportion of water is defined as a function of the desired consistency for the resorbable preparation.

Further characteristics and variants are given in the dependent claims.

### BRIEF DESCRIPTION OF THE DRAWINGS

The features of the invention that do not emerge from what has been said previously, are highlighted in the following description, with reference to the accompanying drawings, in which:

FIG. 1 represents schematically a view of a portion of a limb affected from an extensive injury and a dressing prepared according to the invention;

FIG. 2 represents a schematic view, taken along the sectional plane II-II of FIG. 1, of a dressing prepared according to the invention, in the case of extensive but shallow injury;

FIG. 2a represents a schematic view, taken along the sectional plane II-II of FIG. 1, of a dressing prepared according to the invention, in the case of a deeè injury;

FIG. 3 is a schematic view illustrating the use of the preparation according to the invention as a portion of a catheter.

### DESCRIPTION OF PREFERRED EMBODIMENTS OF THE INVENTION

According to the invention, a preparation was developed particularly and primarily intended for use as dressing in the treatment of difficult healing wounds, These are extensive superficial wounds, burns, deep wounds with involvement of muscle bundles, undermined wounds etc.

In particular, the preparation has the characteristic of consisting of components that can be absorbed by the body and is in the form of amorphous hydrogel composed of a mixture containing at least four main components. These components work synergistically with an adjuvant action for the injured tissue reconstitution, for hydration of the same and protection from pathogens. The four components are made up, in different and variable proportions depending on the use, by: water; non-hydrolyzed, animal or synthetic collagen; glycerol; ozonized vegetable oil. In any case, for predictable uses of the preparation, the concentration of collagen is preferably between 40% and 70% by weight of the complete preparation.

To obtain the resorbable preparation, the non-hydrolyzed collagen, in the form of sheets or powder, is hydrated by mixing it with water to the product maximum solubility temperature (collagen is one of the most hydrophilic proteins, and therefore its hydration can take place without further operations).

Glycerol is then added the mixture in a proportion of 20% to 50% by weight, and preferably in a proportion of 38% by weight of the complete preparation. Glycerol is in liquid phase at room temperature, and possesses a high degree of solubility in water.

The formulation of the resorbable preparation according to the invention, in its most general form, is then completed with the addition of ozonized vegetable oil, in proportion of 0.001% to 10% by weight, preferably in proportion of 2% by weight of the complete preparation. As it is insoluble in water, the ozonized vegetable oil is mixed to the preparation with suitable stirring, so as to obtain a fine emulsion and to distribute the oil in a substantially homogeneous manner in the hydrogel thus obtained.

The vegetable oil ozonation is a known and commonly used stabilization technique of ozone, that has very short half-life in the free state (in the order of seconds). For example, there are known applications in products for cosmetics; in sport, where it is used for massage as an aid in reducing and metabolization of lactic acid; and in the treatment of chronic forms in rheumatology.

Ozone binds to the double bonds of the oil fatty acids, forming compounds called ozonides and thus remaining trapped and available to the tissues when applied topically. With the known procedures, ozone concentrations can be obtained up to 60-70%, sufficient to dispense continuously up to 2,000 peroxides/ml.

In particular, for the formulation of resorbable preparation of the invention, the use of ozonized sunflower oil is preferred.

Optionally, xylitol can be added to the mixture in proportion of 0.005% to 5% as well as lactoferrin, in proportion of 0.001% to 10%.

In particular, xylitol has recognized bactericidal and biofilm inhibition properties in the injured area.

Also lactoferrin, a glycoprotein antimicrobial and iron-transporter, typical of milk and found in various mucosal secretions (saliva, tears), efficiently hinders the growth of the biofilm.

It should be considered that, for the purposes of the invention, the sequence of mixing of the components can be arbitrarily varied without that, however, the final result of the operation is modified.

The preparation so obtained is suitable for many uses, according to which it can be defined and substantially shaped according to bi- or three-dimensional structures.

In general, hydrogels more or less compact can be obtained by adjusting the concentration of the various components, and especially their dilution in water.

For example, a abundantly hydrated, and thus more “liquid”, formulation of the resorbable preparation allows the use thereof in a 3D printer to realize a kind of gauze with a desired thickness and shape, or for making tubular elements with predefined diameter and length. A less hydrated formulation can be advantageously used to realize the same devices by means of conventional plastic molding techniques. The uses of such devices will become apparent in the following description.

Due to its peculiar characteristics, the hydrogel formulation has the advantage of allowing a precise definition of the consistency of the resorbable preparation, at the time of preparation of the hydrogel, or during the realization of the devices, or later on during their use in applications that will be described in the following.

Basically, the hydrogel formulation allows a precise control, in addition to the initial consistency, also on the subsequent rehydration mechanisms. A very hydrated hydrogel is preferable when a constant moisture would preferably be provided to the implant site, while a more compact and drier hydrogel is required if the device is to provide good mechanical strength and must retain its three-dimensional structure for longer time (as it takes longer to rehydrate).

In general, the three major components of the resorbable preparation in hydrogel present functions and purposes that in part are compensated and in part are strengthened by the presence of more than one component. These functions will be hereinafter described with reference to the main, but not exclusive, applications of resorbable preparation of the invention.

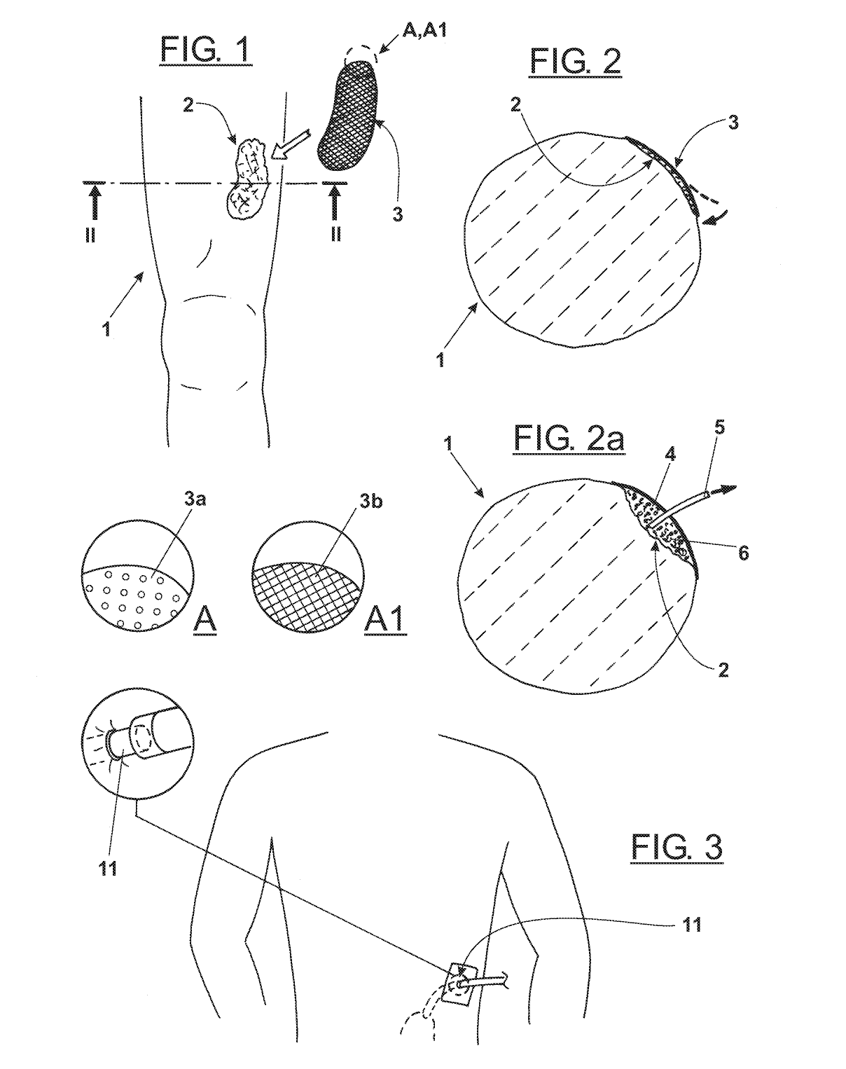

First, the preparation which has an elected use as dressing in the treatment of difficult wounds, which have a high extension and/or affect deep tissues, not only those limited to the dermis but also the ones extending to the underlying muscle bundles. In this regard, reference will be made to FIGS. 1, 2 and 2a, and to the details A and A1 of the drawing in FIG. 1.

In this application, the resorbable preparation can be prepared in the form of compact sheet or gauze, with predefined thickness, placed in contact with the injured area tissue being formed, subsequently to the operations of cleansing and asepsis of the area itself.

FIG. 1 illustrates a limb 1 affected by a major injury 2. This injury 2 is superficial in FIG. 2, while it is deeper in FIG. 2a.

A dressing 3 in the form of resilient sheet, as a kind of gauze, made with resorbable preparation, subject-matter of the invention, is applied to the wound 2, so as to adapt perfectly to the surface conformation of the wound.

The gauze can be realized in different ways, for example with a perforated sheet structure 3a (see particular A of FIG. 1) or with a reticular structure (especially A1 of FIG. 1), but also, if desired, as a compact sheet.

As previously mentioned, the dressing 3 can be prepared at the time of use by means of a 3D printing starting from the liquid or semi-liquid preparation, with the desired consistency. Alternatively, it can be previously prepared and packaged to be subsequently used. In this case, the production consistency may be already the desired one, or it may be less hydrated, and thus more “dry” and rigid. Then the preparation is rehydrated before use until it gets the consistency required by the application.

The dressing 3, particularly if prepared in situ by 3D printing, can easily be shaped according to the shape and extent of the wound 2 to medicate, even if this has an irregular or complex shape (for example, it is shaped like a hand or ear if the injury extends totally over these parts, or in the form of a wrapping bandage, or any other).

Depending on the required degree of hydration, the final composition of the dressing 3 may have the following proportions of the various components:
water: from 10% to 90% by weight of the complete preparation;
collagen: from 40% to 70% by weight of the complete preparation;
glycerol: from 20% to 50% by weight of the complete preparation;
xylitol: from 0.001% to 5% by weight of the complete preparation;
ozonized sunflower oil: from 0.001% to 10% by weight of the complete preparation;
lactoferrin, in proportion in the range of 0.001% to 10% by weight.

The proportion of water in the preparation at the time of preparing the dressing defines the characteristics of compliance. A very hydrated preparation is soft and conformable; while a less hydrated is more rigid.

In general, the degree of hydration of the dressing at the time of its use can be defined in several ways. For example; if it is carried out immediately before use, a preparation can be used that has a proportion of water substantially equal to the required degree of hydration. Alternatively, the dressing can be prepared earlier, and then subjected to drying up to obtaining a stiffer consistency but mechanically more resistant, and therefore more manipulable; finally, it can be packed for later use. At the time of use, the dressing can finally be extracted from the package, re-hydrated to the desired degree by soaking it in water, and then applied on the wound.

As already mentioned, in addition to the advantages given by the possibility of shaping the dressing 3 as desired, the resorbable preparation has several properties particularly useful in facilitating healing of the wound 2, properties provided by the components of the preparation itself.

Collagen has the capability, already known and recognized, to promote granulation of the tissue in formation. Furthermore, it is one of the components of the new cells and extracellular matrix, and is the main component of the dermis. It can then be used during the reconstitution process of damaged tissues, thus avoiding production and transportation thereof to the area of the wound by the organism. Basically, a simply absorbent dressing, as the traditional ones, is replaced with a dressing 3 which is also able to give himself to the full within the wound. This allows a speedup of the healing time.

The dressing is also able to act as “alternative target” for the matrix metalloproteases during the proliferation process, thus preserving the collagen newly formed by the wound fibroblasts.

Furthermore, since collagen is highly hydrophilic, the dressing 3 complies with the requirements for the dressing of wounds in a moist environment, which, as it was shown, allows a healing rate many times higher than a medication in a dry environment.

The function of the glycerol in the resorbable preparation is twofold. In the first place, it promotes hydration of the gel and retention of the water absorbed by the collagen; therefore, it prevents excessive crystallization and maintain good conformability of the dressing 3. Secondly, the glycerol has a recognized bacteriostatic function, and therefore helps to prevent the development of infections in the wound.

Xylitol, as already mentioned, offers bactericidal capacity and contributes to inhibit the formation and proliferation of the bacterial biofilm.

Also the ozonized vegetable oil plays a promoting action of granulation and, given the presence and continuous release of ozone, has a strong antiseptic power and inhibits the formation and proliferation of the biofilm in the injured area.

FIG. 2a illustrates a deep wound 2, treated with the negative pressure therapy technique. According to this technique, an air-impermeable sheet 4 is applied to the wound 2; the sheet extends beyond the edges of the wound and is made to adhere to the non-injured skin that surrounds it. A small tube 5 is passed through the sheet 4 and is connected to a vacuum source, to constantly keep a vacuum in the injured area and contextually drain excess exudate.

A dressing 6, prepared with the resorbable preparation according to the invention, has three-dimensional spongy structure and is placed inside the sheet 4. The dressing 6 can be advantageously obtained immediately before its application by means of 3D printing, and can take the exact shape of the space comprised between the wound 2 and the impermeable sheet 4.

Also in this case the dressing 6 can be prepared in advance, possibly dried, and packaged; then, it will be subsequently extracted from the package and, if necessary, rehydrated before being applied on the wound.

In this way the advantages deriving from the negative pressure therapy dressing technique can conveniently be associated to those due to the presence of a dressing 6 in a moist environment, which provides the body with material to replenish the injured tissues. This dressing performs a considerable antiseptic action and does not need to be replaced, but is progressively resorbed by the body itself with the progress of tissue reconstitution.

Another advantageous effect of the resorbable preparation used as a medication for wounds is that it blocks the action of the metal-protease (as already mentioned, the enzyme complexes involved in removing the “debris” of infection, namely the matrix extracellular). The metalloprotease ensure local degradation of the extracellular matrix, to allow specialized cells, such as leukocytes, to pass through it. This function, that is is very useful in limiting the inflammatory processes, also gets the unintended effect of delaying, when not to prevent, the closure and the correct healing of a superficially exposed wound, thus causing a chronic wound and therefore it facilitates still more the healing.

A further example of use of the invention, shown in FIG. 3, consists of a tubular, rigid or semi-rigid element 11, intended to be used as a resorbable catheter in temporary catheterizations.

Also in this case, the catheter 11 can either be obtained by means of a 3D printing based on the implant requirements, or previously produced and packaged, to be opened immediately prior to use.

The catheter 11, according to the shown application, is connected to the outside with a traditional drainage tube. The advantage, as compared to conventional catheterizations, is given by the fact that, once completed its function, the catheter 11 has not be removed, but is progressively reabsorbed.

In the latter application the hydration of the hydrogel preparation is such as to provide the resorbable catheter 11 with adequate mechanical strength characteristics for this use and the expected duration of the catheterization. Since the collagen is gradually rehydrated at the implant site and reabsorbed by the body, an initially more hydrated hydrogels is more flexible and less durable than one initially less hydrated and therefore more rigid.

In any case, the proportion of water initially contained in the hydrogel of the catheter 11 prior to implantation may vary, for example, from 10% to 90% and by weight of the resorbable preparation of which it consists. The catheter 11, before implantation, may also be subjected to hydration or drying operations, according to the mechanical characteristics required for use.

In a last, but not exhaustive example of application of the resorbable preparation of the invention, the preparation is shaped as a plate for ostomy and associated with a relative pocket, for use in case of intestinal or urinary ostomy.

Currently, resins derived by acid abietic (pine resin) in paste form are used for attaching the plate to the skin around the stoma, which can cause allergies. In fact, the plaque consists of a circular structure with an adhesion ring, which is bonded to the skin by means of adhesive pastes based on rosin, a resin containing abietic acid.

At least one active substance can also be associated to the resorbable preparation of the invention, for example an broad-spectrum antiseptic or an antibiotic, that is intended to increase infection prevention action of the medical-sanitary device made with the preparation.

The preparation can also act as a carrier for any other drug that is necessary to lead directly into the injury site, to facilitate healing.

Depending on the type of active ingredient, this drug may for example be associated to the aqueous fraction, if water-soluble, or the lipid fraction of the resorbable preparation, if fat-soluble.

It is understood that the described apparatus can be used, making appropriate variations which, however, fall within the inventive concept in question, also in other areas and sectors, without departing from the spirit of the invention.

It is also understood that different embodiments or variations of the present apparatus falling within the protection scope of the present invention as described above and defined in the following claims.

1. A resorbable preparation for medical use, characterised by including an amorphous hydrogel obtained from a mixture of animal collagen, glycerol and ozonized vegetable oil in water.

2. A resorbable preparation in accordance with claim 1, characterised in that said mixture is composed of water in a proportion ranging from 10% to 90% by weight, collagen in a proportion ranging from 40% to 70% by weight, glycerol in a proportion ranging from 20% to 50% by weight, and ozonized vegetable oil in proportion ranging from 0.001% to 10% by weight; the proportion of water being defined as a function of the desired consistency for the resorbable preparation.

3. A resorbable preparation in accordance with claim 1, characterised in that said collagen consists of not hydrolyzed collagen, in the form of sheets or powder.

4. A resorbable preparation in accordance with claim 1, characterised in that said ozonized vegetable oil consists of ozonized sunflower oil.

5. A resorbable preparation in accordance with claim 1, characterised by including at least a hydrosoluble active principle, dissolved in the aqueous fraction of said preparation.

6. A resorbable preparation in accordance with claim 1, characterised by including at least a fat-soluble active principle, dissolved in the lipid fraction of said preparation.

7. A resorbable preparation in accordance with any one of claim 1, characterised by further including a part of xylitol, in a proportion ranging from 0.05% to 5%.

8. A resorbable preparation in accordance with claim 1, characterised by further including a part of lactoferrin, in a proportion ranging from 0.001% to 10%.

9. The use of a resorbable preparation in accordance with claim 1, as a dressing for difficult wounds, wherein said dressing is made in form of a flexible sheet, adapted to take the conformation of the wound to be covered.

10. The use of a resorbable preparation in accordance with claim 9, wherein said flexible sheet is in reticular form or uniformly perforated.

11. The use of a resorbable preparation in accordance with claim 1, as a dressing for difficult wounds, wherein said dressing is made in the form of a flexible three-dimensional structure with spongy appearance, adapted to fill recesses present in wounds and to match the shape of the same, the water proportion in said preparation being in the range of 10% to 90% by weight.

12. The use of a resorbable preparation in accordance with claim 9, wherein said sheet or three-dimensional structure is obtained by 3D printing.

13. The use of a resorbable preparation in accordance with claim 9, wherein said sheet is obtained by molding.

14. The use of a resorbable preparation in accordance with claim 1, as a resorbable catheter in temporary catheterizations, wherein said preparation is shaped as a rigid or semi-rigid tubular element.

15. The use of a resorbable preparation in accordance with claim 1, as a plate for ostomy, wherein said preparation is shaped in the form of a concave or convex drilled pad, associated with an ostomy bag.
